(19) United States
(12) Patent Application Publication
(10) Pub. No.: US 2012/0053122 A1
(43) Pub. Date: Mar. 1, 2012

(54) METHOD FOR THE PROPHYLAXIS OR TREATMENT OF FLUSHING

(75) Inventor: Johannes Mathijs Maria de Boer, Velp (NL)

(73) Assignee: Maatschap Interne Geneeskunde Rijnstate

(21) Appl. No.: 13/145,762

(22) PCT Filed: Jan. 19, 2010
(86) PCT No.: PCT/NL2010/050027

§ 371 Date: Sep. 28, 2011

Related U.S. Application Data
(60) Provisional application No. 61/146,419, filed on Jan. 22, 2009.

(51) Int. Cl. A61K 38/09; A61P 5/04

(52) U.S. Cl. 514/10.4; 514/10.3

(57) ABSTRACT

The present invention relates to a method for the prophylaxis or treatment of a subject for flushing, wherein a pharmaceutical agent having LHRH antagonistic activity is administered to said subject at a dosage of less than 15 μg/kg/day. The present invention also relates to a pharmaceutical agent having LHRH antagonistic activity for use in the prophylaxis or treatment of a subject for flushing, wherein the pharmaceutical agent having LHRH antagonistic activity is administered to said subject at a dosage of less than 15 μg/kg/day. The flushing is preferably postmenopausal flushing or perimenopausal flushing.

### FIELD OF THE INVENTION

The present invention relates to a method for the prophylaxis or treatment of flushing in a subject, wherein a pharmaceutical agent having LHRH antagonistic activity is administered to said subject.

### BACKGROUND OF THE INVENTION

Flushing (or “hot flashes”, “hot flushes” or “night sweat”) is a symptom of the decrease in gonadal hormone levels that occurs around menopause or andropause. Flushing may start to appear several years before the onset of menopause and may last for years afterwards. While some menopausal women never have hot flashes, others suffer from flushing many times a day. Where menopause is caused by surgery or medical treatment, flushing is often more severe.

Nighttime flushing may disrupt normal sleep. This can severely affect the quality of life since it often causes psychological and physical problems (e.g. fatigue, depressive mood, impaired concentration).

Flushing is not limited to women. Male patients having low testosterone levels may also suffer from flushing. However, flushing is most commonly observed in women.

Postmenopausal (PMP) flushing is the most frequent somatic complaint for which women in their climacterium seek medical help. Unfortunately, its exact pathophysiological background remains incompletely understood (D. W. Sturdee, Maturitas 2008; 60: 257). This lack of knowledge hampers the development of new drugs to treat severe flushing. PMP flushing clearly is an estrogen dependent phenomenon, its onset is related to estrogen deficiency or withdrawal, and its treatment is most effectively done by estrogen replacement therapy (ERT). However, with the knowledge of today, patients and doctors are increasingly reluctant to use ERT for PMP flushing symptoms because it may increase the risk of cardiovascular disease, stroke, thrombo-embolism, and cancer of the breast and endometrium. In breast cancer survivors estrogen treatment for hot flushes is contraindicated because it is associated with a three-fold increased risk of a new primary or a breast cancer recurrence.

About 30% of women in natural menopause, and about 50% of breast cancer survivors have severe PMP flushing that considerably affects their quality of life. The more severe degree of flushing in breast cancer patients is related to the abrupt onset of menopause caused by chemotherapy or surgery. It is further aggravated by the use of estrogen receptor blockers and aromatase inhibitors. Up to 20% of breast cancer patients consider stopping adjuvant endocrine therapy because of its aggravating effects on flushing.

In the past 15 years several drugs have been tested to treat PMP flushing in order to find a substitute for ERT. However, all these treatments are only moderately effective, and none are without side effects. The most potent agents are paroxetine, venlafaxine and gabapentin. They are about twice as effective as placebo treatment, whereas estrogen treatment is four times as effective and reduces PMP flush frequency by 80-90%. Consequently, paroxetine (a SSRI-antidepressant), venlafaxine (a SNRI-antidepressant) and gabapentin are not very effective.

U.S. Pat. No. 6,703,367, incorporated by reference herein, discloses a method for treating hot flashes and gynaecomastia, wherein a subject in need thereof is treated with a LHRH antagonist. The hot flashes may be the result of a series of disorders or treatments. The LHRH antagonist may be parenterally or orally administered or it may be administered as a controlled release formulation. The LHRH antagonist may be selected from a large group of agents, e.g. the compound which is identified as PPI-149 (disclosed in U.S. Pat. No. 5,843,901) which is the most preferred agent. The dosage of the LHRH antagonist covers a range of 15-300 μg/kg/day. All examples of U.S. Pat. No. 6,703,367 refer to the treatment of hot flashes in males which are the result of therapies for prostate cancer.

US 2005/250793, incorporated by reference, discloses a method of preventing or treating hot flashes with a medicament which comprises a non-peptidic compound having gonadotropin releasing hormone antagonistic activity.

### SUMMARY OF THE INVENTION

The present invention relates to a method for the prophylaxis or treatment of a subject for flushing, wherein a pharmaceutical agent having LHRH antagonistic activity is administered to said subject at a dosage of less than 15 μg/kg/day. The present invention likewise relates to a method for the prophylaxis or treatment of flushing in a subject, wherein a pharmaceutical agent having LHRH antagonistic activity is administered to said subject at a dosage of less than 15 μg/kg/day. The present invention also relates to a pharmaceutical agent having LHRH antagonistic activity for use in the prophylaxis or treatment of flushing in a subject, wherein the pharmaceutical agent having LHRH antagonistic activity is administered to said subject at a dosage of less than 15 μg/kg/day.

### DESCRIPTION OF THE FIGURES

FIG. 1: Patient 1. Shortterm in-hospital response in to Cetrorelix 250 μg once a day for 5 days (left column) and 6 months out-patient response to Cetrorelix 250 μg once a day. The gray area represents active treatment. Bottom figures, open dots: total daily flush frequency, filled dots: nighttime flush frequency (counted between 23.00 h and 07.00 h).

FIG. 2: Patient 2. Shortterm in-hospital response to Cetrorelix 250 μg once a day (left column). Longterm out-patient response to Cetrorelix 250 μg once a day, followed by Cetrorelix 250 μg twice a day (right column). Grey area represents active treatment, arrow and interrupted line indicate start of Cetrorelix twice a day). Open dots: total daily flushes, closed dots: nighttime flushes.

FIG. 3: Patient 3. Shortterm (left column) in-hospital response (right column) to Cetrorelix 250 μg once a day for 5 days, followed by Cetrorelix 250 μg twice a day for 5 days, and out-patient response to Cetrorelix 500 μg once a day for 1 month (right column). The grey area represents active treatment. Left column, diamond filled dots: LH and FSH measurement at 8.00 h, open dots: LH and FSH measurement at 20.00 h. Bottom figures, open dots: total daily flushes, filled dots: nighttime flushes.

### DETAILED DESCRIPTION OF THE INVENTION

### Definitions

The verb “to comprise” as is used in this description and in the claims and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

In this document, the term “flushing” is to be understood as including the synonyms “hot flashes”, “hot flushes” and “night sweat”. The term “flushing” is also to be understood as including physiological conditions related to a secondary type of flushing (sometimes also referred to as “slow hot flashes” or “ember flashes”). Normal flushing comes on rapidly and may reach its maximum intensity within a minute. The maximum intensity will last only for several minutes before it gradually fades. The secondary type of flushing also appears rapidly, but is less intense and last for a longer period of time, e.g. thirty minutes.

Additionally, the term “pharmaceutical agent having LHRH antagonistic activity” is intended to include LHRH antagonists, also known as GnRH antagonists, in its broadest sense. Consequently, this term not only includes compounds typically known in the art as LHRH receptor antagonists, but also inactivators or deactivators of natural LHRH, suppressors of LHRH secretion or LHRH production or both. GnRH antagonists have a mechanism of action which is very different from GnRH agonists. Some important pharmacological actions are described in U.S. Pat. No. 4,800,191 and U.S. Pat. No. 5,198,533. In particular, GnRH agonists are similar to the neurohormone GnRH and interact with the gonadotropin-releasing hormone receptor to elicit its biologic response, i.e. the release of the pituitary hormones FSH and LH. Since GnRH agonists do not rapidly dissociate from the GnRH receptor, there is initially an increase in FSH and LH secretion (“flare effect”). However after about ten days, a profound hypogonadal effect (i.e. a decrease in FSH and LH secretion) is achieved through receptor downregulation by internalization of receptors. Generally this induced a reversible hypogonadism, which is the therapeutic goal. On the other hand, a GnRH antagonist competes with the neurohormone GnRH for GnRH receptor binding and decreases the secretion of FSH and LH.

### Dosages and Methods of Administration

According to the present invention, the pharmaceutical agent having LHRH antagonistic activity is administered at a dosage of less than 15 μg/kg/day. This implies that e.g. at a body weight of 70 kg, the dosage is less than about 1 mg (=1000 μg) per day.

It is preferred that the dosage is less than or equal to about 14 μg/kg/day, more preferably less than or equal to about 12 μg/kg/day, even more preferably less than or equal to about 10.5 μg/kg/day, and most preferably less than or equal about 9 μg/kg/day. At a body weight of 70 kg, this implies that the dosage is preferably less than or equal to about 900 μg per day, more preferably less than or equal to 800 μg per day, even more preferably less than or equal to about 700 μg per day and most preferably less than or equal to about 600 μg per day.

The minimum dosage is preferably 0.75 μg/kg/day, more preferably 1.0 μg/kg/day, even more preferably 1.5 μg/kg/day and most preferably 3.75 μg/kg/day. At a body weight of 70 kg, this implies that the minimum dosage is preferably about 50 μg per day, more preferably about 67 μg per day, even more preferably about 100 μg per day, yet even more preferably about 175 μg per day and most preferably about 250 μg per day. According to an embodiment of the present invention, the pharmaceutical agent having LHRH antagonistic activity is administered in a daily dosage of about 100 μg to less than about 1000 μg per day, more preferably of about 100 μg to about 900 μg, even more preferably about 100 μg to about 800 μg, yet even more preferably about 100 μg to about 700 μg and yet even more preferably about 100 μg to about 600 μg.

The pharmaceutical agent having LHRH antagonistic activity can be administered in various ways, e.g. subcutaneously, orally, intranasally, intravenously, intradermally, or intramuscularly. However, according to the invention, the pharmaceutical agent is preferably administered subcutaneously, most preferably by injection of a liquid formulation comprising the pharmaceutical agent and a liquid pharmaceutically acceptable carrier.

On the other hand, the pharmaceutical agent having LHRH antagonistic activity may also be administered via a device for controlled release, e.g. a (infusion) pump, a transdermal patch or an implant.

According to the invention, the pharmaceutical agent having LHRH antagonistic activity may be administered intermittently or continuously.

According to a intermittent administration scheme, the pharmaceutical agent is preferably administered for 2 to 10 days, preferably for 3 to 8 days, where after the administration is interrupted for 5 days to six weeks, preferably 5 days to three weeks, more preferably, 5 days to 10 days.

According to a continuous administration scheme, the pharmaceutical agent is preferably administered every day during the first three (e.g. Monday, Tuesday and Wednesday; no administration on Thursday, Friday, Saturday and Sunday) to first five days (e.g. Monday, Tuesday, Wednesday, Thursday and Friday; no administration on Saturday and Sunday) of the week for a period of 1-12 months, preferably 2-10 months.

Alternatively, in a continuous administration scheme, the pharmaceutical agent may be administered daily, every other day, once per week, once per month, once per 3 months or longer intervals extending to once a year. The total period of administration may vary, but is preferably within a period of 1-12 months, preferably 2-10 months.

According to the present invention, the preferred administration scheme is daily, every other day, once per week or once per month.

### Pharmaceutical Agent

According to the present invention, it is preferred that the pharmaceutical agent having LHRH antagonistic activity is selected from the group consisting of oligopeptides, wherein the oligopeptide comprises 2-14 amino acid residues. More preferably, the oligopeptide comprises 6-12 amino acid residues and even more preferably 8-12 amino acid residues. Most preferably, the oligopeptide comprises 9-11 amino acid residues.

It is preferred that the oligopeptides are selected from the group consisting of LHRH antagonists. The LHRH antagonists are preferably selected from the group consisting of Abarelix (CAS. No. 183552-38-7; also known as Plenaxis), Antarelix (Teverelix; CAS No. 144743-92-0), A-775998 (CAS No. 135215-95-1), Ganirelix (CAS No. 123246-29-7, 129311-55-3 and 124904-93-4; also known as Antagon), Azaline B (CAS No. 134457-28-6 and 214766-78-6), Detirelix (CAS No. 89662-30-6), Ramorelix (CAS No. 127932-90-5 and 136639-71-9), Degarelix (CAS No. 214766-78-6), Cetrorelix (CAS No. 120287-85-6; also known as Cetrotide), Ozarelix (CAS No. 295350-45-7) and RS-68439 (CAS No. 102583-46-0). Most preferably, the LHRH antagonist is Cetrorelix.

### Patients

As described above, flushing may occur in male and female subjects. However, according to the invention, the subject is female. It is further preferred that the subjects are mammals, most preferably humans.

Preferably, the method according to the invention is preferably directed to the treatment of postmenopausal flushing or perimenopausal flushing. Most preferably, the flushing is the (partly) result of the treatment for breast cancer, ovary cancer or both.

### Pharmaceutical Compositions

The present invention also relates to a pharmaceutical agent having LHRH antagonistic activity for use in the prophylaxis or treatment of flushing, wherein said pharmaceutical agent being preferably administered to said subject at a dosage of less than 15 μg/kg/day. Preferred dosage ranges and dosage regimens are described above. Preferred embodiments of the pharmaceutical agent having LHRH antagonistic activity are also described above.

### EXAMPLES

### Patients and Methods

Three women who were referred for treatment of severe PMP flushing were included in the pilot study, after signing an informed consent. All three had frequent and severe flushing, during daytime as well as during the night. Sleep was often disturbed by flushes and sweat attacks, often forcing a change of clothes and/or bed linen. Physical examination in these patients was unremarkable and other causes of flushing were excluded. Thyroid function was normal, 24 hour urinary hydroxy-indol-acetic acid and catecholamines were within the reference range, serum tryptase was normal, and none used any medication known to induce flushing. All three had used clonidine previously, without experiencing a significant benefit.

### Patient 1

Patient 1 was 65 years. At the age of 44 she underwent a hysterectomy and bilateral ovariectomy. Postoperatively she started ERT. This was discontinued at the age of 50. Since then she had severe PMP flushing which was most problematic during the night. Each night she awakened 3-4 times because of severe flushes accompanied by fierce perspiration and palpitations. A change of pyjamas up to twice a night occurred regularly. During daytime she had at least 6 flushes, and this number doubled during hot summer days. Clonidine up to a dose of 75 μg twice a day had been tried but had no beneficial effect. Medical history further included mild hypertension since 2 years, for which she was treated successfully with lisinopril. Physical examination was normal, body weight was 83 kg with a body mass index (BMI) of 32.0 kg/m².

### Patient 2

Patient 2 was 49 years at the time of referral. At the age of 46, breast cancer was diagnosed in the right breast (T2N1M0, estrogen and progesterone receptor positive). At that time she was still premenopausal. She underwent a unilateral mastectomy and several weeks later both ovaries were removed laparoscopically. Subsequently she started adjuvant endocrine therapy with tamoxifen 20 mg once a day, and after 2 years she was switched to anastrazole 1 mg/day, according to protocol. Flushing had started on the first day after oophorectomy and it rapidly grew worse. She slept poorly because of frequent night flushes and sweat attacks, about 3-4 each night. Total daily flush frequency varied from 15 to 30. Clonidine 50 μg twice a day had no beneficial effect on flushing. Physical examination was unremarkable, body weight was 72 kg and BMI 28.8 kg/m². Clonidine was gradually withdrawn 6 weeks before she entered the study. Anastrazole was continued at 1 mg/day, taken before breakfast.

### Patient 3

Patient 3 was 49 years of age. Fifteen years previously she was successfully treated for Crohn's disease, and now she was in remission for 8 years, and without anti-inflammatory medication. She did use quinalapril for mild hypertension. Eighteen months previously an abdominal hysterectomy and bilateral ovariectomy was performed because of endometrium carcinoma stage I. Immediately after surgery she developed severe flushing, about 16 times during the day, and about 4 times during the night. As a result, sleep quality was poor. She was easily agitated which was attributed to chronic fatigue caused by the frequent and severe sleep disturbances. Clonidine 75 μg twice a day had no effect whatsoever. At the time of testing she only used quinalapril for mild hypertension and pantozole for pyrosis. Physical examination was normal, except for marked obesity. Body weight was 106 kg, BMI 39.8 kg/m².

### Methods

To avoid intermittent and variable interference of external factors as much as possible, and to guarantee a constant ambient temperature all subjects were hospitalized for further study. They remained ambulatory during that period. They were instructed to record the following flush characteristics in a diary: time of onset, duration in minutes, flush severity on a scale of 0-10, and the degree of associated sweat attacks: 0=no sweating; 1=mild sweating, i.e. only moist skin, no visible sweat drops; 2=moderate sweating, i.e. visible sweat drops; 3=severe sweating, i.e. totally wet, change of clothes necessary. Blood samples for measurement of serum LH, FSH, estradiol, SHBG and albumin were taken each day at 08.00 h and 20.00 h.

LH, FSH and estradiol were measured by electrochemiluminescene immunoassays (Roche Diagnostics, Mannheim, Germany). Normal ranges in PMP women: LH>10 U/L, FSH>30 U/L, estradiol<100 nmol/L. SHBG was measured by chemilumiescent enzyme immunoassay (DPC, Los Angeles, Calif., USA, normal range in PMP women: 18-144 nmol/L)).

### Example 1

### Short-Term Responses, In-Hospital Setting

### Patient 1

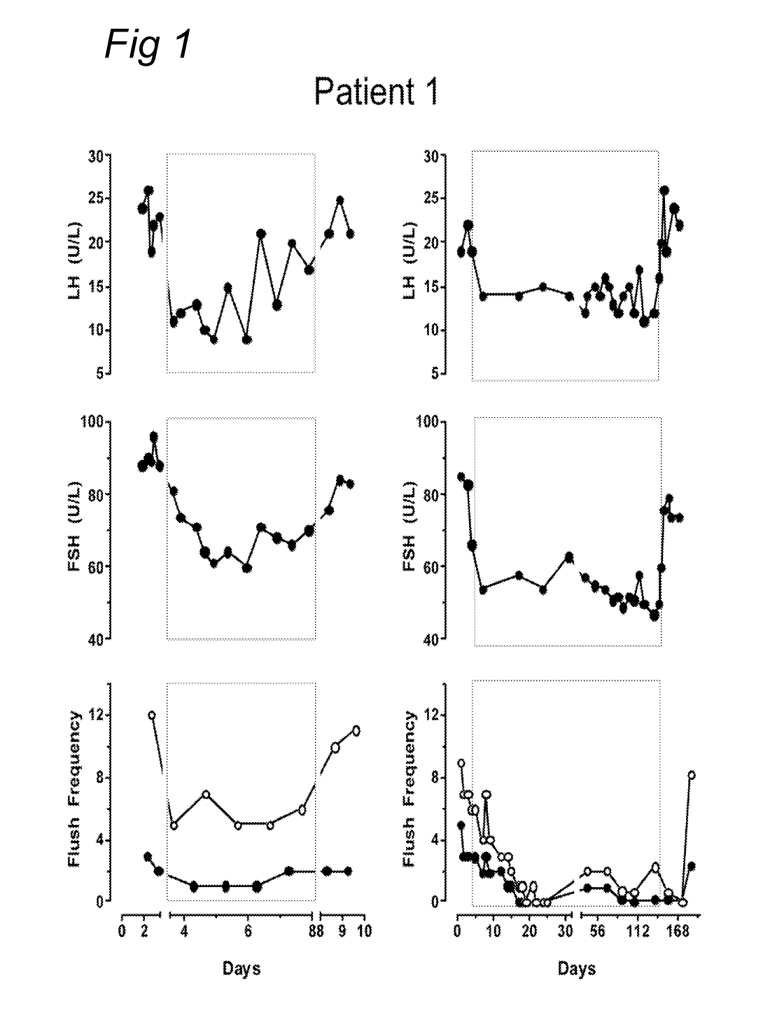

After an observation period of two nights and one day, Cetrorelix 250 μg was given once a day for five days. Serum LH levels decreased abruptly from about 22 to 10 U/L within 12 hours (FIG. 1, left column). The change in serum FSH was more gradual and decreased from about 90 to 60 U/L in 3 days. LH levels subsequently tended to rise in a sawthoothed pattern, starting from the 3^{rd} day of Cetrorelix administration. Serum estradiol, SHBG and albumin levels did not change during treatment. Total flush frequency, daytime flush frequency and nighttime flushing decreased abruptly by 60-70%, within 24 hours, and remained around these new levels during the next treatment days. Nighttime flush duration tended to decrease, daytime flush duration did not change appreciably. Flush severity showed a weak trend to decrease, in particular during daytime. After discontinuation of Cetrorelix serum LF, FSH and flush frequency rose again to pre-treatment levels within 48 hours.

### Patient 2

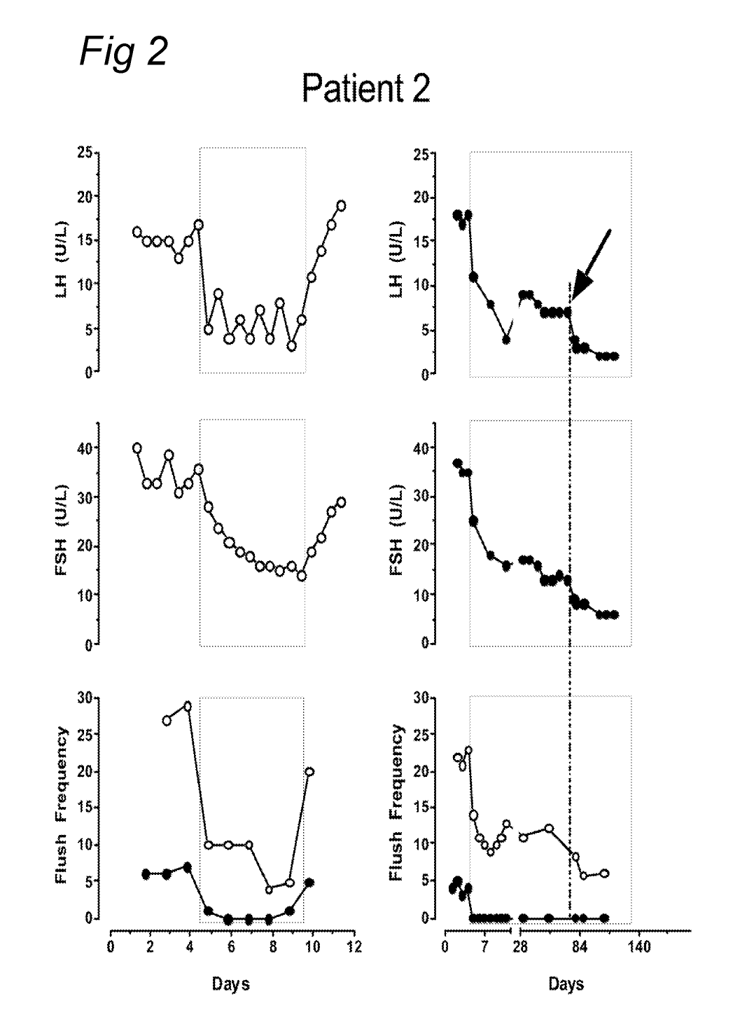

After a pretreatment observation period of 3 nights and 2 days, Cetrorelix 250 μg was started, once a day. The abrupt decrease in serum LH within 12 hours and the gradual decrease in serum FSH level that occurred after the start of treatment were comparable to the responses observed in patient 1 (FIG. 2, left column). FSH reached its nadir later than in patient 1, i.e. at 5 days instead of 3 days. Daytime flush frequency decreased within one day from 20 to 8 (−60%) and then gradually fell to 4 flushes per day at the 4^{th} day of treatment (−80%). Nighttime flushing decreased from 5 to 1 on the first night of treatment (−80%), and completely disappeared thereafter. The patient reported that for the first time in years she had slept undisturbed, without awakenings. Daytime flush duration tended to decrease slightly, daytime flush severity and flush sweating did not change significantly. After discontinuation of Cetrorelix LH returned to pre-treatment levels within 48 hours, FSH rose somewhat more slowly. Flush frequency increased but did not reach pre-treatment levels within the two days after the discontinuation of Cetrorelix.

### Patient 3

After a 3-day pre-treatment observation period Cetrorelix was started, in dose of 250 μg once a day, administered at 08.30 h. Again, LH decreased abruptly, and FSH more gradually.

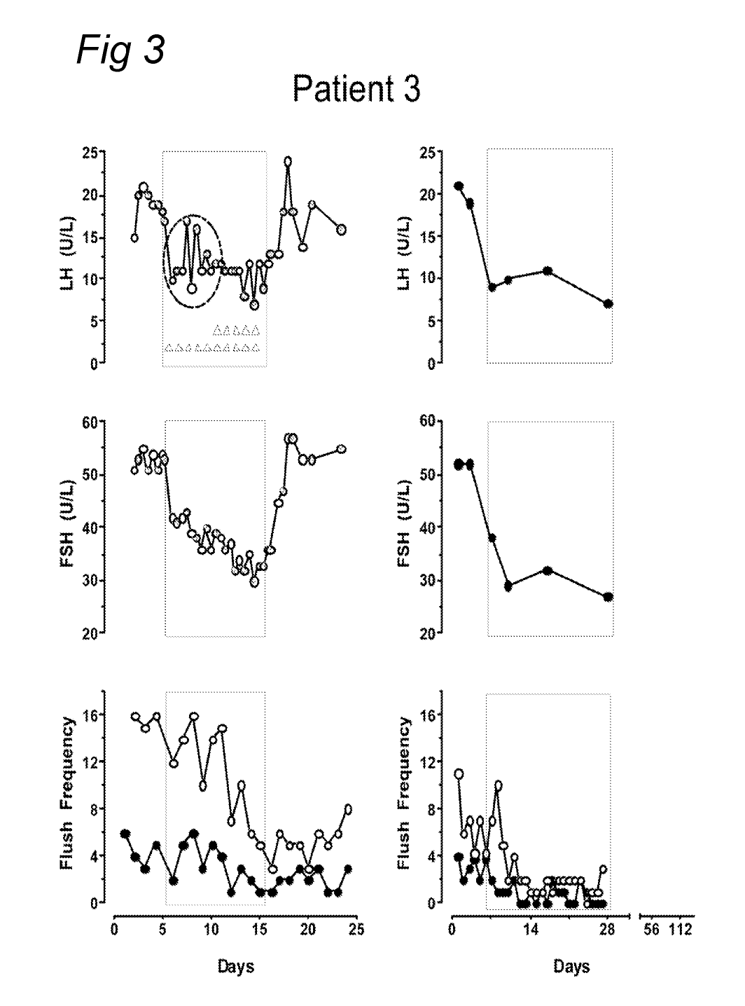

On the second day of treatment morning elevations of LH levels were observed, suggesting incomplete LHRH receptor blockade (FIG. 3, left column, diamond filled dots). However, on the following days the morning LH peaks gradually decreased, without a change in Cetrorelix dosage. FSH continued to decrease gradually and showed no escape phenomena. After the first 5 days of treatment there was only a small decrease in total and daytime flushes (−25%), whereas nighttime flushing did not change significantly. It was concluded that this patient might be undertreated, and therefore the dose was increased to 250 μg twice a day. Now, nighttime flushing also started to decrease. Flush frequency remained decreasing until one day after the discontinuation of Cetrorelix. At that time total daily flush frequency had decreased by 80%. After discontinuation of treatment LH and FSH rose rapidly as observed before. In contrast to the observations in patient 1 and 2, it now took several days before flushing started to return to pretreatment levels. Six days after discontinuation of Cetrorelix the flush frequency was still markedly lower than before treatment. Thereafter, a gradual rise to pre-treatment levels was observed.

### Example 2

### Long-Term Treatment, Outpatient Setting

To evaluate whether responsiveness was maintained in the long run, Cetrorelix treatment was studied for a prolonged period in an outpatient setting. Cetrorelix was started after a baseline observation period of 3-6 days. In the first two patients the starting dose was of 250 μg once a day, injected at about 8.30-9.00 h, just after the morning blood sampling. In the third patient the starting dose was 500 μg once a day. Blood was sampled only in the morning, between 8.00 en 9.00 h, every day during the first 4 days, thereafter every 3 days for a brief period, and later on once a week.

### Patient 1

Serum LH and FSH decreased to levels similar to those observed during the in-hospital setting (FIG. 1, right column). Pretreatment daytime flush frequency was lower, and nighttime flush frequency was higher that in the hospital setting. In contrast to the abrupt response in hospital, the day- and nighttime flush frequencies now decreased gradually and after two weeks treatment they were close to zero. Flush severity gradually decreased from about 8 to 4, during the day as well as during the night. If flushes occurred, their duration and the degree of perspiration were unchanged.

To evaluate whether long-term LHRH receptor blocking was reversible, Cetrorelix was discontinued after 4 months. Blood sampling and flush recording was intensified. Within 3 days LH and FSH levels rose to pretreatment levels. Flush frequency started to rise 6 weeks after the discontinuation of treatment.

### Patient 2

Within a week after the start of treatment serum LH had decreased to premenopausal levels, whereas FSH stabilized around 15 U/L, i.e. just above the upper limit for premenopausal women. Daytime flush frequency decreased from about 18 to 8 flushes/day within the first week, and nighttime flushing disappeared completely within two days. Over the following weeks daytime flush duration, severity and perspiration gradually decreased also. Because daytime flushing eventually levelled off at 10 flushes/day, it was decided to increase Cetrorelix to 250 μg twice a day after the third month of treatment. Blood sampling and flush monitoring were intensified temporarily to assess the changes more precisely. Serum LH and FSH levels and flushing further decreased. Eventually total daily flushing decreased to 6 flushes/day, i.e. a total decrease of about 75%.

### Patient 3

Because the in-hospital testing had shown that 250 μg was not sufficiently effective, this patient was started on Cetrorelix 500 μg once a day, administered at 20.00 h. During the first 4 weeks serum LH and FSH decreased to a nadir of 8 and 25 U/L, respectively. Flush frequency decreased from 9.67±1.12 (mean±standard error of the mean) to 2.00±0.44 (decline of 79.3%, P<0.007).

1-23. (canceled)

24. A method for the prophylaxis or treatment of flushing in a subject, comprising administering to a subject in need thereof less than 15 μg/kg/day of a pharmaceutical agent having LHRH antagonistic activity.

25. The method according to claim 24, wherein the pharmaceutical agent comprises an oligopeptide.

26. The method according to claim 25, wherein the oligopeptide comprises 2-14 amino acid residues.

27. The method according to claim 24, wherein the pharmaceutical agent is selected from the group consisting of Abarelix, Antarelix (Teverelix), A-775998, Ganirelix, Nal-Glu antagonist, Azaline B, Detirelix, Ramorelix, Degarelix, Cetrorelix, Ozarelix and RS-68439.

28. The method according to claim 24, wherein the subject is female.

29. The method according to claim 24, wherein the pharmaceutical agent is administered subcutaneously.

30. The method according to claim 24, wherein the pharmaceutical agent is administered intermittently or continuously.

31. The method according to claim 24, wherein the subject is postmenopausal or perimenopausal.

32. The method according to claim 24, wherein the flushing is the result of breast cancer treatment.

33. The method according to claim 24, wherein the flushing is the result of ovary cancer treatment.

34. A pharmaceutical composition comprising less than or equal to about 900 μg of a pharmaceutical agent having LHRH antagonistic activity.

35. A method for the prophylaxis or treatment of flushing in a postmenopausal or perimenopausal female subject, comprising administering to the subject in need thereof less than 15 μg/kg/day of a pharmaceutical agent having LHRH antagonistic activity.
